(19) United States
(12) Patent Application Publication
(10) Pub. No.: US 2025/0213462 A1
(43) Pub. Date: Jul. 3, 2025

(54) NOVEL PHOTONIC CRYSTAL PROTEINS FROM ATRINA SP. BYSSUS AND USES THEREOF

(71) Applicant: POSTECH RESEARCH AND BUSINESS DEVELOPMENT FOUNDATION, Pohang-si (KR)

(72) Inventors: Dong Soo Hwang, Pohang-si (KR); Ji Min Choi, Pohang-si (KR)

(21) Appl. No.: 19/002,887

(22) Filed: Dec. 27, 2024

(30) Foreign Application Priority Data
Dec. 27, 2023 (KR) 10-2023-0192743
Dec. 26, 2024 (KR) 10-2024-0196892

(51) Int. Cl. A61K 8/98; A61K 8/02; A61K 8/64; A61Q 19/00; A61Q 19/08

(52) U.S. Cl. CPC A61K 8/987; A61K 8/027; A61K 8/64; A61Q 19/007; A61Q 19/08

(57) ABSTRACT

The present invention relates to a novel photonic crystal protein derived from Atrina sp. byssus and its use. According to the present disclosure, seasilk, which is processed to have a specific color using the byssus of Atrina sp., has a less impact on the marine ecosystem in terms of utilization of waste resources and a high economic value and thus can be actively used as eco-friendly and functional fibers and cosmetic materials.

### CROSS-REFERENCE TO RELATED APPLICATION

This present application claims the benefit of priority to Korean Patent Application No. 10-2023-0192743, entitled “Novel photonic crystal proteins from Atrina sp. byssus and uses thereof,” filed on Dec. 27, 2023, and Korean Patent Application No. 10-2024-0196892 of the same title, filed on Dec. 26, 2024, in the Korean Intellectual Property Office, the entire disclosure of which is incorporated herein by reference.

### FIELD

The present disclosure relates to novel photonic crystal proteins derived from Atrina sp. byssus and uses thereof.

### BACKGROUND

Natural silk protein materials have been used as a representative clothing material for a long time. Recently, the natural silk protein materials have very high applicability as a next-generation high-functional industrial material due to characteristics such as excellent biocompatibility of natural silk proteins, outstanding elasticity and physical strength, the ability to be molded into various forms such as powder, membrane, porous body, and gel, and the ability to be chemically modified as a natural protein.

In addition, the natural silk proteins are functional materials with various physiological activities, and are effective as an energy source for energy metabolism by containing abundant essential amino acids, monosaccharides, various vitamins, and minerals, and are also known to be rich in antioxidants and have excellent effects in regulating cell growth and metabolism, and thus have been used in various cosmetics.

Accordingly, efforts have been made to search for new natural silk protein materials existing in nature and to first secure original substance patents for new silk protein materials.

In the 2000s, it has been found that among them, silk proteins had a distinct difference in properties depending on producing species, and silk-like proteins with similar structures and sequences to conventional silks or new structures and sequences existed in insects such as spiders, bees, ants, dragonflies, and grasshoppers, and marine organisms such as mussels and shrimp. However, the insects and marine organisms may not be bred, and the amount of silk proteins obtained is minimal, making practical use impossible.

Meanwhile, the marine organisms have the characteristic of adhering to surfaces by methods such as byssus, rhizomes, mucous substances, and suction, in order to survive in dynamic marine environments. Among them, seasilk, as fabrics made from the byssus of Pinna nobilis in the family Pinnidae, is a fiber that has been regarded as a luxury fiber in Western culture since the 4th century AD due to toughness, lightness, warmth, and golden color with a natural luster to have a high economic value and has been used by humans for a long history. Fabrics produced from the seasilk have an advantage (ultrathin) of being woven finer and thinner than silkworm silk or spider silk, and has an ultralight property. Due to its excellence as the fiber and aesthetic factors, although the seasilk has a high economic value since the beginning of recorded human history, due to the significant reduction in the population of Pinna species, their capture is legally prohibited in the EU, and then legal harvesting and production are severely limited to have limitations on the production of seasilk. In addition, research on the production of silk from marine species is almost non-existent due to the low level of exploration at the genetic and protein levels of species that live in the ocean, and thus its components are still unclear and only fragmentary information on its structure has been found. Meanwhile, the silk has been required for various colors, especially a luxury golden color, and various methods have been developed to meet the demand, and the development of golden seasilk is expected to lead to a rapid increase in demand in the silk industry.

In addition, technologies for developing new materials and developing highly functional cosmetics have been continuously developed across the current cosmetics industry.

Therefore, as an alternative for replacing Pinna sp. byssus, which is a traditional raw material that had produced seasilk, the discovery of new seasilk and species having the new seasilk may solve a problem of discovering new high value-added materials, which is facing the development of conventional natural clothing and cosmetic materials.

### SUMMARY

Under this situation, the present inventors found for the first time through amino acid composition analysis, molecular biological analysis, and nanostructure analysis that the Pinna sp. byssus, which had been previously used as a raw material for seasilk, and the Atrina sp. byssus exhibited the same characteristics. Then, the present inventors found that when silk fiber derived from Atrina sp. byssus obtained therefrom was acid-treated, color tuning to a specific color and skin improvement efficacy were excellent by photonic crystal proteins derived from Atrina sp. byssus, and then completed the present disclosure.

Therefore, the object of the present disclosure is to provide a method for producing a golden seasilk fiber derived from Atrina sp. byssus.

Another object of the present disclosure is to provide a composition for producing a golden seasilk fiber.

Yet another object of the present disclosure is to provide a functional cosmetic composition for improving skin, including a seasilk fiber derived from Atrina sp. byssus; or a golden seasilk fiber derived from Atrina sp. byssus as an active ingredient.

Other objects and advantages of the present disclosure will be more apparent by the following detailed description and claims.

The terms used herein are used for the purpose of description only, and should not be construed to be limited. A singular expression includes a plural expression unless otherwise clearly defined differently in a context. In the present disclosure, it should be understood that term “including” or “having” indicates that a feature, a number, a step, an operation, a component, a part or the combination thereof described in the specification is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise clearly defined in the present disclosure.

Hereinafter, the present disclosure will be described in detail.

According to one aspect of the present disclosure, the present disclosure provides a method for producing golden seasilk fiber derived from Atrina sp. byssus, including an acid treatment processing step of treating a seasilk fiber derived from Atrina sp. byssus, which is isolated from Atrina sp. byssus, with an acid solution, and a composition for producing a golden seasilk fiber including a golden seasilk fiber derived from Atrina sp. byssus produced according to the method.

In the present disclosure, the seasilk fiber derived from Atrina sp. byssus includes a photonic crystal Photonin-1 protein derived from Atrina sp. byssus, consisting of an amino acid sequence as set forth in SEQ ID NO: 1, and a photonic crystal Photonin-2 protein derived from Atrina sp. byssus, consisting of an amino acid sequence as set forth in SEQ ID NO: 2, and is used interchangeably with sea fiber (sea silk) in the present specification.

The term ‘photonic crystal’ refers to a material with a lattice period having a length similar to the wavelength of light, which means a material that has a structure capable of using optical properties of the material, or is made to have the structure.

The range of the Photonin-1 and Photonin-2 proteins according to the present disclosure includes proteins having amino acid sequences as set forth in SEQ ID NOs: 1 and 2, respectively, and functional equivalents of the proteins. The “functional equivalent” has sequence homology of at least 70% or more, preferably 80% or more, more preferably 90% or more, much more preferably 95% or more with the amino acid sequences as set forth in SEQ ID NOs: 1 and 2, as a result of the addition, substitution or deletion of the amino acid and refers to a protein having substantially homogeneous physiological activity with the protein as set forth in SEQ ID: 2. The “substantially homogeneous physiological activity” refers to a photonic crystal property.

The seasilk fiber derived from Atrina sp. byssus of the present disclosure becomes a golden seasilk fiber having a golden structural color that does not fade due to a change in longitudinal repeating structure induced by acid treatment.

In addition, the golden seasilk fiber derived from Atrina sp. byssus has a hierarchical structure stabilized by sugar-lectin interactions and has a structural color.

In addition, according to the present disclosure, the present disclosure provides genes encoding the Photonin-1 and Photonin-2 proteins, respectively.

In addition, homologs of the base sequences of the genes encoding the proteins according to the present disclosure are included within the scope of the present disclosure. Specifically, the genes may include base sequences having at least 70%, preferably at least 80%, more preferably at least 90%, and most preferably at least 95% of sequence homology with the base sequences encoding the proteins as set forth in SEQ ID NOs: 1 and 2, respectively. The “% of sequence homology” with the polynucleotide is confirmed by comparing two optimally aligned sequences with a comparison region, and a part of the polynucleotide sequence in the comparison region may include addition or deletion (i.e., gap) compared to a reference sequence (without addition or deletion) for an optimal alignment of the two sequences.

In addition, according to the present disclosure, the present disclosure provides a method for producing a photonic crystal Photonin protein derived from Atrina sp. byssus, including overexpressing a gene encoding the photonic crystal Photonin protein by transforming a host cell with a recombinant vector including the gene.

The term “recombinant” refers to a cell that replicates a heterologous nucleic acid, expresses the nucleic acid, or expresses a protein encoded by a peptide, a heterologous peptide or a heterologous nucleic acid. The recombinant cell may express a gene or gene fragment that is not found in a natural form of the cell in either sense or antisense form. In addition, the recombinant cell may express genes found in cells in the natural state, but the genes have been modified and reintroduced into the cell by artificial means.

In the present disclosure, the Photonin-1 and Photonin-2 proteins; or gene sequences encoding the Photonin-1 and Photonin-2 proteins may be inserted into a recombinant expression vector. The term “recombinant expression vector” means bacterial plasmid, phage, yeast plasmid, plant cell virus, mammalian cell virus, or other vectors. In general, any plasmid and vector may be used as long as the plasmid and vector may replicate and stabilize in the host. Important characteristics of the expression vector have an origin of replication, a promoter, a marker gene and a translation control element.

The expression vector including the Photonin-1 and Photonin-2 proteins or the gene sequence encoding the proteins and suitable transcription/translation control signals may be constructed by methods well known to those skilled in the art. The methods include in vitro recombinant DNA technology, DNA synthesis technology, in vivo recombination technology, etc. The DNA sequence may be effectively linked to a suitable promoter within an expression vector to lead mRNA synthesis. In addition, the expression vector may include a ribosome binding site as a translation initiation site and a transcription terminator.

A method for delivering the vector of the present disclosure into a host cell may be performed by a CaCl₂) method, a Hanahan method (Hanahan, D., J. Mol. Biol., 166:557-580 (1983)), an electroporation method, and the like when the host cell is a prokaryotic cell. In addition, when the host cell is a eukaryotic cell, the vector may be injected into the host cell by a gene gun-mediated transformation method (bombardment), an Agrobacterium-mediated transformation method, a microinjection method, a calcium phosphate precipitation method, an electroporation method, a liposome-mediated transfection method, a DEAE-dextran treatment method, and the like, but is not limited thereto.

The host cell is not limited as long as the purpose of the present disclosure may be achieved, but may be at least one selected from the group consisting of, for example, E. coli, yeast, animal cells, plant cells, and insect cells.

Further, the present disclosure provides a method for producing a seasilk fiber derived from Atrina sp. byssus, including spinning a composition for preparing a seasilk fiber derived from Atrina sp. byssus including the Photonin-1 and Photonin-2 proteins described above, that is, at least one amino acid sequence selected from the group consisting of amino acid sequences as set forth in SEQ ID NOs: 1 and 2, respectively.

The spinning method may use any method known in the art as long as the purpose of the present disclosure may be achieved.

In addition, according to the present disclosure, native seasilk fibers directly separated from Atrina sp. byssus are used.

In an embodiment of the present disclosure, the native byssus itself, which is collected from Atrina sp. by cutting the byssus, was used as a seasilk fiber. Atrina sp. is a large shellfish and is cultivated for edible purposes, but the byssus of Atrina sp. is not an edible resource. Therefore, the seasilk fiber derived from Atrina sp. byssus of the present disclosure by using the byssus has great significance in terms of utilization of waste resources.

According to the present disclosure, the seasilk fiber derived from Atrina sp. byssus of the present disclosure has a hierarchical structure stabilized by sugar-lectin interactions and has a structural color. In addition, a color change is enabled with a structural color that is irreversibly fixed by an external stimulus such as an acid, and preferably, the color change may be controlled and implemented to a gold color in various spectra by controlling pH.

In general, the ‘color’ of an object is a color of the wavelength of light reflected from visible light reaching the object, and such a color includes a pigment color caused by pigments and a structural color caused by the diffraction, interference, and scattering phenomena of light. When the object with the structural color is magnified with an electron microscope, a regular arrangement appears, and when light is flashed thereon, only light of a predetermined wavelength is reflected and the rest is transmitted. Such a geometrical shape is called a photonic structure, and the photonic structure has a regular arrangement and spreads out in a three dimension, which is called a photonic crystal.

Therefore, the present inventors found a photonic fiber having photonic crystals in which the Atrina sp. byssus has a fade-proof golden color without a dye and exhibits green fluorescence in the absence of light through amino acid composition analysis, molecular biological analysis, and nanostructural analysis. In addition, the present inventors found that the fade-proof golden color is caused by the structural color through fiber structural analysis (TEM, AFM, AI-based structural analysis, small angle X-ray diffraction, wide angle X-ray diffraction), molecular biological methods (NGS, ESI-LC-MSMS, immunohistochemistry), and reflection microscopy analysis.

In the present disclosure, the acidic solution may be added to an aqueous system to result in a pH of less than 7, and may be used with any acid known in the art as long as the purpose of the present disclosure may be achieved. For example, the acidic solution may be used with at least one selected from the group consisting of sodium citrate, acetic acid, boric acid, sorbic acid, citric acid, sodium phosphate, dibasic sodium phosphate, monobasic sodium phosphate, potassium dihydrogen phosphate, hydrochloric acid, sodium hydroxide, sodium thiosulfate, sodium sulfite, sodium sulphate, tris(hydroxymethyl)aminomethane, tris(hydroxymethyl)aminomethane hydrochloride, sodium hydrogen carbonate, sodium borate, sodium acetate, sodium bisulphate and sodium benzoate, but is not limited thereto.

In an embodiment of the present disclosure, in order to implement a conventional lemon juice treatment method, 0.3 M sodium citrate at pH 1.5 to 2.0 was treated, and it was demonstrated that color changes of gold in various spectra may be controlled and implemented by adjusting the pH.

In addition, in the present disclosure, the acid treatment may be used with at least one selected from the group including lemon juice, citron juice, sugar citron juice, orange juice, magnolia berry juice, bokbunja juice, wolfberry juice, cornelian cherry juice, plum juice, and vinegar.

In addition, the acid treatment is not limited to an immersion ratio as long as the purpose of the present disclosure may be achieved, and for example, the seasilk fiber and the acidic solution may be immersed at a ratio of 1:1000 to 1000:1 (w/w).

In addition, according to yet another aspect of the present disclosure, the present disclosure provides a functional cosmetic composition for improving skin, including the seasilk fiber derived from Atrina sp. byssus described above; or a golden seasilk fiber derived from Atrina sp. byssus as an active ingredient.

The seasilk fiber derived from Atrina sp. byssus and the golden seasilk fiber derived from Atrina sp. byssus include a photonic crystal Photonin-1 protein consisting of an amino acid sequence as set forth in SEQ ID NO: 1 and a photonic crystal Photonin-2 protein consisting of an amino acid sequence as set forth in SEQ ID NO: 2.

The seasilk fiber derived from Atrina sp. byssus is transformed into a golden seasilk fiber with a fade-proof golden structural color by inducing a change in longitudinal repeating structure through acid treatment, and the golden seasilk fiber derived from Atrina sp. byssus has a hierarchical structure stabilized by sugar-lectin interactions and has a structural color.

According to the present disclosure, since the composition exhibits growth-promoting activity of human skin fibroblasts, the composition may exhibit at least one skin improvement effect selected from the group consisting of improved skin moisturizing, improved skin wrinkles, improved skin thickness, improved skin elasticity, improved skin barrier, improved skin texture, and improved pigmentation.

In addition, when the composition of the present disclosure is prepared with the cosmetic composition, the composition of the present disclosure may include ingredients commonly used in the cosmetic composition as well as the above-described active ingredients, and may include, for example, conventional auxiliary agents such as antioxidants, stabilizers, solubilizers, vitamins, pigments, and fragrances, and carriers.

The cosmetic composition may be prepared in any formulation known in the art as long as the purpose of the present disclosure may be achieved, and for example, the formulation may be at least one selected from the group consisting of a toner, a nourishing toner, a massage cream, a nourishing cream, a pack, a gel, a skin-adhesive type cosmetic, a lipstick, a makeup base, a foundation, a shampoo, a rinse, a body cleanser, a soap, a lotion, an ointment, a gel, a cream, a patch, and a spray, but is not limited thereto.

That is, the cosmetic composition of the present disclosure may be prepared even in any formulation commonly prepared in the art, and may be formulated by, for example, a solution, a suspension, an emulsion, a paste, a gel, a cream, a lotion, a powder, a soap, a surfactant-containing cleansing, oil, a powder, a foundation, an emulsion foundation, a wax foundation, a spray, and the like, but is not limited thereto. More specifically, the cosmetic composition of the present disclosure may be prepared in formulations of a toner, a nourishing toner, a nourishing cream, a massage cream, an essence, an eye cream, a cleansing cream, a cleansing foam, cleansing water, a pack, a spray or a powder.

When the formulation of the present disclosure is the paste, cream, or gel, as the carrier ingredient, animal oils, vegetable oils, wax, paraffin, starch, tragacanth, cellulose derivatives, polyethylene glycol, silicone, bentonite, silica, talc, zinc oxide or the like may be used.

When the formulation of the present disclosure is the solution or emulsion, as the carrier ingredient, a solvent, a solubilizing agent or an emulsifying agent may be used. For example, the carrier ingredient includes water, ethanol, isopropanol, ethyl carbonate, ethyl acetate, benzyl alcohol, benzyl benzoate, propylene glycol, 1,3-butyl glycol oil, glycerol aliphatic ester, polyethylene glycol or fatty acid ester of sorbitan.

When the formulation of the present disclosure is the suspension, as the carrier ingredient, a liquid diluent such as water, ethanol or propylene glycol, a suspension such as ethoxylated isostearyl alcohol, polyoxyethylene sorbitol ester and polyoxyethylene sorbitan ester, microcrystalline cellulose, aluminum metahydroxide, bentonite, agar, tragacanth, or the like may be used.

When the formulation of the present disclosure is the powder or spray, as the carrier ingredient, lactose, talc, silica, aluminum hydroxide, calcium silicate or polyamide powder may be used, and particularly, in the case of the spray, a propellant such as chlorofluorohydrocarbon, propane/butane or dimethyl ether may be further included.

When the formulation of the present disclosure is the surfactant-containing cleansing, as the carrier ingredient, aliphatic alcohol sulfate, aliphatic alcohol ether sulfate, sulfosuccinic acid monoester, isethionate, imidazolinium derivatives, methyltaurate, sarcosinate, fatty acid amide ether sulfate, alkylamido betaine, aliphatic alcohol, fatty acid glyceride, fatty acid diethanolamide, vegetable oil, lanolin derivatives, ethoxylated glycerol fatty acid ester, or the like may be used.

The cosmetic composition of the present disclosure may be used alone or in combination, or may be used in combination with other cosmetic compositions other than the present disclosure. In addition, the cosmetic composition according to the present disclosure may be used according to a conventional using method, and the number of uses may vary depending on the user's skin condition or preference.

When the cosmetic composition of the present disclosure is the soap, the surfactant-containing cleansing formulation, or the surfactant-free cleansing formulation, the cosmetic composition may be applied to the skin and then wiped off, removed, or washed with water. As a specific example, the soap includes liquid soap, powder soap, solid soap and oil soap, the surfactant-containing cleansing formulation includes cleansing foam, cleansing water, cleansing towel and cleansing pack, and the surfactant-free cleansing formulation includes cleansing cream, cleansing lotion, cleansing water and cleansing gel, but are not limited thereto.

Therefore, as long as the composition of the present invention achieves the intended effect, it is expected that the seasilk derived from Atrina sp. byssus of the present disclosure is used as a raw material for a functional cosmetic composition to provide a skin functional cosmetic suitable for a subject.

Since the method of the present disclosure uses the composition of the present disclosure described above, the description of common contents therebetween will be omitted in order to avoid excessive complexity of the present specification.

According to the present disclosure, seasilk, which is processed to have a specific color using the byssus of Atrina sp., has a less impact on the marine ecosystem in terms of utilization of waste resources and a high economic value and thus can be actively used as eco-friendly and functional fibers and cosmetic materials.

### BRIEF DESCRIPTION OF THE DRAWINGS

The above and other aspects, features, and advantages of the present disclosure will become apparent from the detailed description of the following aspects in conjunction with the accompanying drawings, in which:

FIG. 1 shows results of comparing amino acid compositions of Atrina byssus and known Pinna byssus;

FIG. 2 shows that the Atrina byssus and the known Pinna byssus are composed of the same protein, and nanostructures formed by the protein have the same size;

FIG. 3 shows that the internal spiral structure of the Atrina byssus is similar to the internal structure of the known Pinna byssus. The peak shapes on small angle X-ray diffraction are the same, and pitch measurements are also 15.57±0.2 for the byssus of Pinna nobilis and 18.21±0.26 for the byssus of Atrina pectinata;

FIG. 4 shows that the internal structure of the Atrina byssus seasilk is formed using sugar-lectin bonds, and that the structure of the sugar-lectin bonds inside the fiber affects the physical properties of the fiber itself. In fibers treated with excessive sugar or lectin, internal bonds that had formed a microfibril structure are broken (D-J) because the conventional sugar-lectin bonds of the fiber are replaced, thereby causing the fiber bundles to disperse (A) and greatly reducing elasticity (B, C). In addition, the sugar that forms bonds with lectin inside the fiber is methyl-D-mannopyranoside;

FIG. 5A shows that the byssus of Atrina sp., from which salt is removed, is treated with lemon juice for 36 hours, in the same manner as a conventional method of producing seasilk, and then turns yellow in the same manner as the seasilk of Pinna byssus. FIG. 5B shows a change in yellow color according to lemon juice or citrate treatment and pH of a citrate solution. Under conditions similar to lemon juice or lemon juice-like 0.3 M citrate (pH 2.0), there is almost no difference in color, and when the pH of citrate is lowered to 1.0, a brighter yellow color is shown;

FIGS. 6 and 7 show changes in a longitudinal repeating structure in Atrina byssus that is changed to gold after lemon juice treatment (q values corresponding to a pitch are divided into 0.0035 A⁻¹, 0.0015 A⁻¹, and 0.0020 A⁻¹). Among these, when q=0.0015 A⁻¹, the pitch of the longitudinal repeating structure is 418.86 nm, and when multiplied by a general refractive index of 1.5 for an organic material, the pitch is 628.30 nm, which matches the wavelength of yellow light;

FIG. 8 shows that after lemon juice treatment, the Atrina byssus reflects a wavelength having a golden color unlike before treatment, and the corresponding wavelength (˜630 nm) matches the wavelength by the converted longitudinal pitch;

FIG. 9 shows cytotoxicity results of seasilk derived from Atrina sp. byssus; and

FIG. 10 shows a skin improvement effect of seasilk derived from Atrina sp. byssus on skin cell lines.

### DETAILED DESCRIPTION

Hereinafter, Examples are to describe the present disclosure in more detail, and it will be apparent to those skilled in the art that the scope of the present disclosure is not limited by these Examples in accordance with the gist of the present disclosure.

### Experimental Methods and Conditions

### Sample Collection

Atrina pectinata was collected from Gangjin, Korea, and the isolated byssus was washed four times with sterile seawater and then used in an experiment.

### Atomic Force Microscopy (AFM)

The surface topology of the byssus washed with sterile water was measured using VEECO Dimension 3100 AFM (Veeco, CA, USA). The measurement conditions were as follows: scan size: 5×5 μm, tapping mode with an Si tip, scan rate: 0.703 Hz. NanoScope Analysis Software (Veeco) was used for 3D imaging.

### Transmission Electron Microscopy (TEM)

For structural analysis of the byssus or acid-treated byssus, a Tecnai G2 transmission electron microscope (FEI, Hillsboro, OR, USA) was used. For observation, grids on which sections of the fiber were mounted were stained with a 2% uranyl acetate solution for 30 minutes, and observed by electron microscopy under 200 kV conditions.

### Separation of Photonic Crystal Proteins

An acid/urea extraction buffer was used for separation of photonic crystal proteins. A fiber washed with sterile water was powdered with liquid nitrogen and homogenized using a homogenizer. A homogenate was centrifuged (15,000×g, 20 min, 4° C.) and the supernatant was collected. The separated protein solution was separated by Tricine-SDS-PAGE (15%) and visualized using a Coomassie Blue solution, and then a protein band area was cut out and the sequence and glycosylation information of the photonic crystal proteins were obtained using an in-gel tryptic digestion method and an ESI-LC-MSMS method.

To identify whether the determined sequence information was actually a structural protein of seasilk, a polyclonal antibody against the following epitope sequence of the corresponding protein was prepared and attached to the fiber: SIHSNKKEDRIFQF (anti-photonin-1), YRIYPEKFYRHLNV (anti-photonin-2), MIDSSGSIGDRPF (anti-Aptmp-2), and GKIKAPNKNERRE (anti-Aptmp-1).

Identification of Structure of Atrina pectinata Byssus Fiber Using Accelerator X-Ray (SAXD/WAXD)

SAXD and WAXD evaluations were performed at a PLS-II 6D UNIST-PAL beamline at Pohang Accelerator Laboratory (PAL), Korea. The energy of X-rays was 11.564 keV for both SAXD and WAXD, and the wavelength was 1.07216 Å. Sample-to-CCD distances for SAXD and WAXD were 3,058.88 mm and 238.02 mm, respectively. The sample-to-detector distance and q-value of SAXD were calibrated using microporous silica, and data were analyzed using a Nika 2D SAS macro package for IGOR Pro (Wavemetrics, Lake Oswego, OR, USA).

### Prediction of Post-Translational Modification (PTM)

An equal amount of sinapinic acid matrix solution (5 mg of sinapinic acid in acetonitrile/water [50/50, v/v] containing 0.1% trifluoroacetic acid) was mixed with the extracted fiber protein solution using the above-mentioned acid/urea buffer extraction method and left on ice for 10 minutes. Thereafter, 1.0 L of the mixed solution was dispensed onto a MALDI plate and the mass was measured. For mass measurement, a BRUKER Autoflex speed LRF Matrix Assisted Laser Desorption/Ionization Time-of-Flight (MALDI-TOF) Mass Spectrometer (Bruker Daltonics, Bremen, Germany) was used.

### Lectin Blotting

The photonic crystal proteins separated by Tricine-SDS-PAGE were blotted onto a polyvinylidene fluoride (PVDF) membrane using a transfer buffer containing 90 mM Tris-borate, 2.5 mM EDTA, 0.1% SDS (w/w), and 25% (v/v) methanol at a constant voltage of 25 V for 90 minutes.

PVDF was washed with TBS-T and then blocked with 5% TBS-T-BSA (1.5 μg/mL) at 4° C. for 12 hours. Subsequently, the membrane was incubated with biotinylated lectin (Vector Laboratories, Burlingame, CA, USA, #BK-2100 #BK-1000) diluted in 5% TBS-T-BSA (1.5 μg/mL) for 1 hour at room temperature, and then washed five times with TBS-T for 5 minutes. Thereafter, the PVDF membrane was incubated with avidin-HRP (eBiosciences, San Diego, CA, USA, #18-4100-51) diluted in 5% TBS-T-BSA (dilution 1:5,000) for 30 minutes and washed five times with TBS-T for 5 minutes again. After washing, protein regions were visualized with an ImageQuant LAS-500 imager system (GE Healthcare Biosciences, Uppsala, Sweden) using an enhanced chemiluminescence (ECL) kit (#170-5060) from Bio-Rad.

Sugar-Lectin Binding in Mechanical Properties of Atrina pectinata Byssus Seasilk

Atrina pectinata byssus seasilk was treated with solutions with or without 0.1 M concanavalin A (Con A) in a citrate buffer (pH 5.5) for 2 weeks, and then changes in threads were observed. For tensile testing, the prepared seasilk was attached to plastic frames with 5 mm spacing and then stored in sterile water for 1 day. All tensile tests were performed at a constant rate (83 μm/s), and an internal humidity chamber and a humidifier were used under wet conditions. In the experiment, a testing device (#Instron 5943, Instron, Norwood, MA, USA) equipped with a 100-N load cell (#2530-100N, Instron, Norwood, MA, USA) was used, and Bluehill software (Instron, Norwood, MA, USA) was used to automatically record data.

Photonic Crystal Properties of Atrina pectinata Byssus Seasilk

To enhance the autofluorescence of Atrina pectinata byssus seasilk, the following experiment was conducted. Seasilk sections cut in a paraffin-blocked state were deparaffinized, and then treated with TBS-T (3% BSA solution, 30 min, 20° C.) to block binding of non-specific proteins, and then incubated with an anti-photonin-1 antibody diluted 1:500 for 2 hours (20° C.). Thereafter, the sections were washed five times with TBS-T for 5 minutes, respectively, and then stained for 1 hour with a Texas Red-conjugated secondary antibody (Thermo Fisher Scientific, Bleiswijk, Netherlands) diluted 1:500 in a blocking solution. Finally, the sections were washed five times with TBS-T for 5 minutes, respectively, and stained slides were photographed using a fluorescence microscope.

The structural colors of seasilk and yellow sea fiber after acid treatment were investigated using a reflection optical microscope Eclipse 80i. The reflection spectrum of the fiber was measured using an optical fiber-coupled spectrometer (Ocean Optics Inc., CA, USA, #HR2000+).

### Cytotoxicity

An MTT assay was performed to evaluate in vitro proliferation of L929 cells. The L929 cells were incubated in Dulbecco's Modified Eagle Medium (DMEM) and after reaching an exponential growth phase, the L929 cells were seeded at a density of 15,000 cells per well in a 96-well plate. After 24 hours of incubation, seasilk solutions with concentrations of 100, 200, 400, and 800 μg/mL were dissolved in the culture solution and treated by two methods:
1) Direct method: The solution containing seasilk particles was directly added to the wells containing cells and a control group (untreated cells) was included.
2) Indirect method: The solution containing seasilk particles was added after pre-incubation for 24 hours in a 37° C., 5% CO₂ environment.

The cells were incubated with a test substance for 24 hours in a 37° C., 5% CO₂ environment. Thereafter, 10 μL of MTT solution (1 mg/mL, PBS) was added per well and further incubated for 4 hours at 37° C. in a dark environment to form purple formazan crystals by living cells. After incubation, the culture solution was removed, 100 μL of DMSO was added to completely dissolve the crystals, and left for 1 hour. The absorbance was measured at 570 nm using a microplate reader. The cell viability was calculated by the following Equation:

$$Cell viability ( \% ) = ( Treated group OD - blank OD ) / ( Untreated group OD - blank OD ) \times 100.$$

### Cell Proliferation

An effect of seasilk particles on the proliferation of fibroblasts (HDFn) was evaluated using a CCK-8 assay. Human dermal fibroblast neonatal (HDFn) cells were cultured in a fibroblast expansion medium supplemented with a low-serum growth kit. The cells were seeded at a density of 1×10⁴ cells per well in a 48-well plate and incubated overnight at 37° C. and 5% CO₂. Then, particle solutions with concentrations of 100, 200, and 400 μg/mL were added to the culture solution, respectively. After 24, 48, and 72 hours of incubation, the culture solution was replaced with a mixture of 100 μL of a fresh culture solution and a CCK-8 reagent (10:1 v/v), and then incubated at 37° C. for 1 hour. Cell viability was calculated using the following Equation:

Cell proliferation(%)=(OD sample−OD Blank)/(OD control−OD Blank)×100

Here, OD sample, OD control, and OD Blank represented the absorbance at 450 nm for an experimental sample, a control (untreated cells), and a blank (culture solution), respectively, and were measured using a microplate reader (Synergy HTX, USA).

### Example 1. Characteristics of Atrina Byssus

Unlike Pinna species, which was designated as a protected species due to a worldwide population decline, Atrina sp. has been widely used as various food resources based on the East Asia, and was an eco-friendly species in terms of waste resource utilization by farming.

Therefore, the present inventors comparatively investigated the compositions and hierarchical structures of the byssi of Pinna nobilis, Mytilus, and Atrina pectinata using molecular biological technique, biochemical analysis, scanning electron microscopy (SEM), transmission electron microscopy (TEM), atomic force microscopy (AFM), wide- and small-angle X-ray diffraction (WAXD/SAXD), and state-of-the-art machine learning-based protein structure prediction.

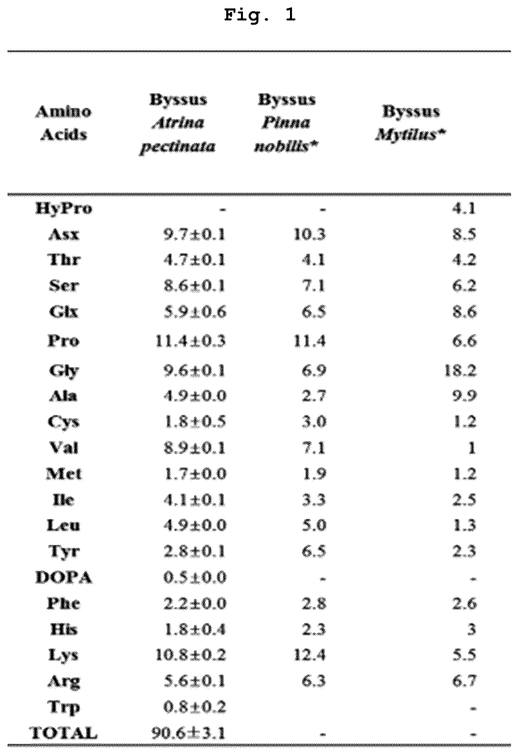

As a result, as shown in FIG. 1, it was confirmed that the amino acid compositions of the Atrina byssus and the Pinna byssus were similar.

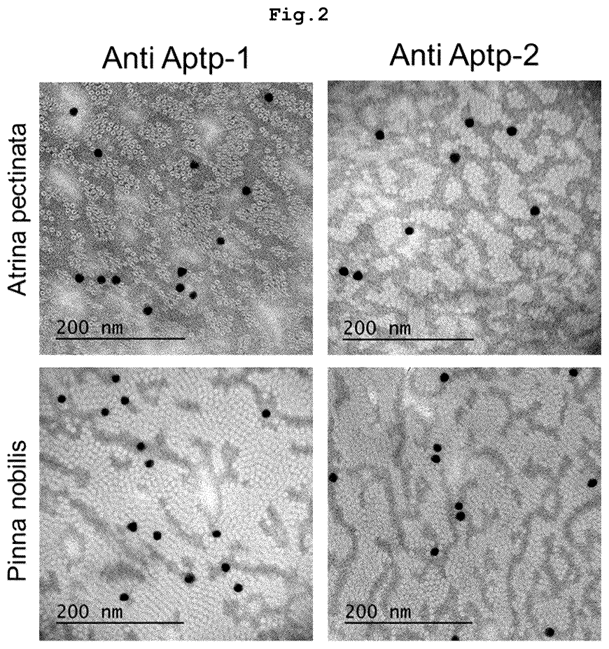

In addition, as shown in FIG. 2, it was confirmed that the Atrina byssus and the Pinna byssus were composed of the same proteins Aptmp-1 and Aptmp-2, and the sizes of the nanostructures formed by the proteins were the same.

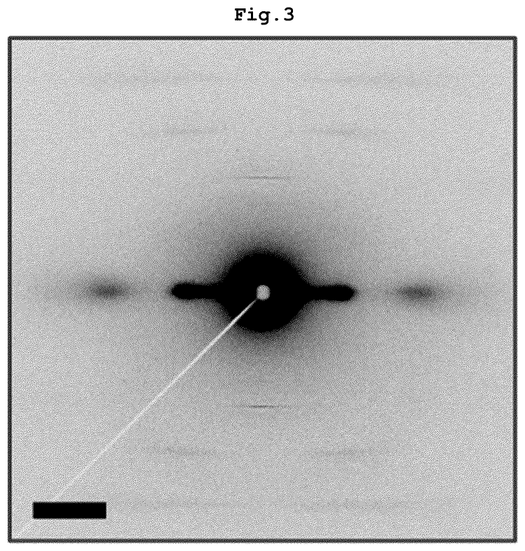

In addition, as shown in FIG. 3, when the internal structure of the Atrina byssus was compared with the internal structure (Delphine et al) of the Pinna byssus known in the art, it was confirmed that the peak shape on small angle X-ray diffraction was identical to that of the Pinna byssus and the measurement value of a pitch was also almost at the same level, which means that the internal spiral structure of the Atrina byssus was identical to the internal structure of the Pinna byssus.

Therefore, the byssus of Atrina pectinata may be used as a substitute for Pinna byssus which is an economically valuable sea fiber.

Furthermore, the present inventors discovered the fact that the seasilk of Atrina pectinata was composed of a hierarchical assembly of globular proteins. Although the configuration does not show a unique secondary structure, an upper hierarchical structure includes the formation of helical nano- and microfibers. In addition, the supramolecular assembly of seasilk is destabilized by lectin treatment, which suggests that the globular protein assembly is cross-linked through sugar-lectin interactions. A hierarchical structure stabilized by sugar-lectin cross-linking strengthens seasilk and enables self-healing. Accordingly, these findings expand the understanding of the hierarchical assembly of natural fibrous materials and provide new insights for designing an engineering material based on sugar-lectin interactions.

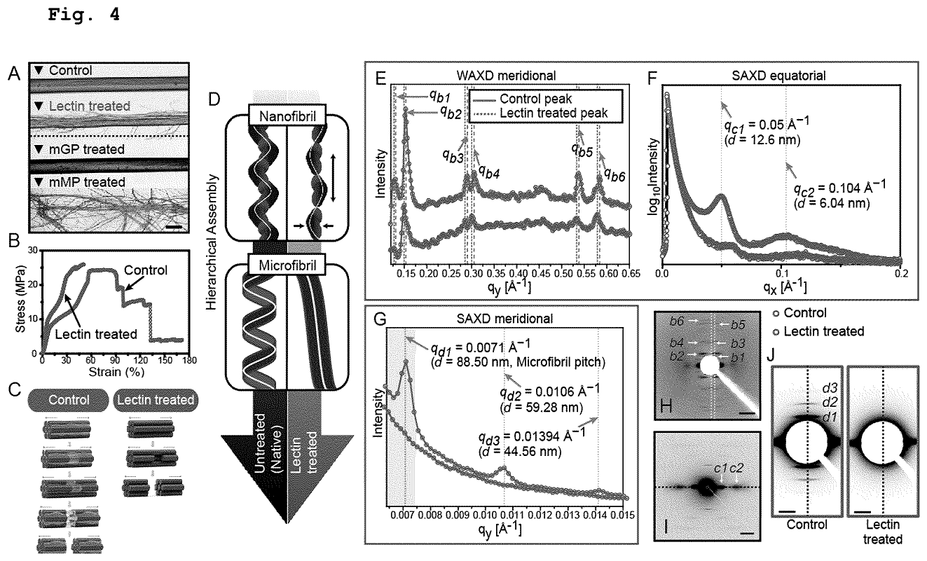

That is, it was confirmed that the seasilk of Atrina pectinata had a distinct hierarchical structure in which globular proteins were assembled into nanofibers and microfibers, and sugar-lectin interactions stabilized the complex structure (FIG. 4).

Furthermore, the hierarchical assembly of globular proteins has been observed even in seasilks of the species Atrina and Pinna in the family Pinnidae, but has also been found in the byssus of the oyster genus Pintada, which is less closely related taxonomically and belongs to a different family.

This suggests that a new structure is not limited to a specific species, but rather may be shared in various species.

Accordingly, a fiber produced by any type of byssus may replace seasilk of Pinna species, as long as the fiber has a structure of a hierarchical assembly of globular proteins in the same manner.

### Example 2. Preparation of Golden Seasilk Fiber Derived from Atrina sp. Byssus

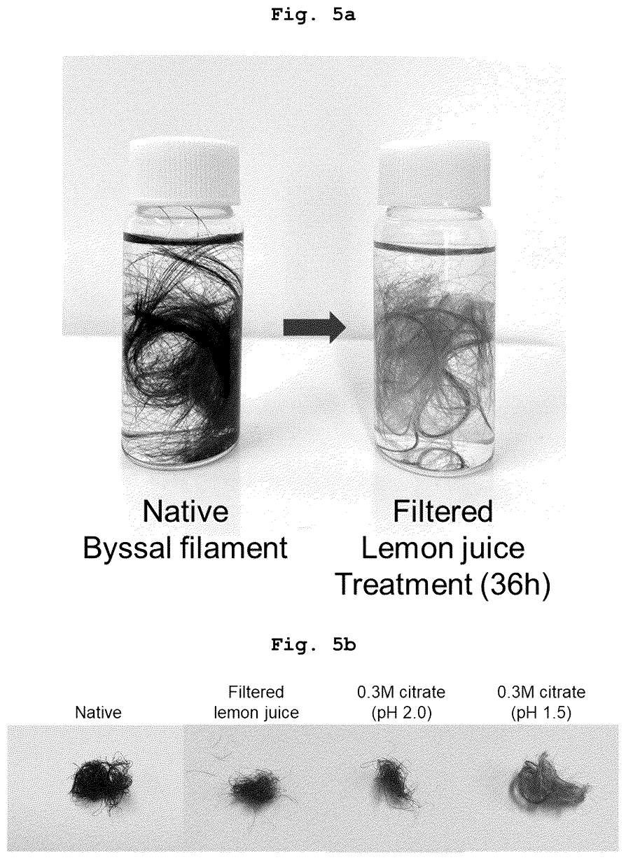

The present inventors obtained fibers from desalinated Atrina sp. byssus, and in order to implement a conventional lemon juice treatment method herein, the fibers were immersed at a ratio of 1 g/10 ml in 0.3 M sodium citrate at pH 2.0 for 36 hours, and then Atrina sp. byssus seasilk fibers with a specific color in various spectra of fade-proof orange to yellow were prepared (FIG. 5A).

In addition, as shown in FIG. 5B, it was confirmed that when the pH decreased to 2.0 or lower, i.e., pH 1.5, the fibers had a lighter color, which proved that various spectra of gold may be implemented by controlling the pH.

In addition, the present inventors confirmed that silk proteins derived from golden Atrina sp. byssus were photonic crystals through optical analysis such as polarizing optical microscopy (POM) and reflection microscopy during this process, which were named Photonins, and these sequences of Photonin-1 (SEQ ID NO: 1) and Photonin-2 (SEQ ID NO: 2) were shown in Table 1.

TABLE 1
Amino acid sequence of Photonin-1
MKNLQHSCLLFLVCSMISYVCEAWNNKYDGLLRFKCKTPQNHIQHILSQHDNKKEDRIYDLRCKKA
TGRRPSCRWTGYVNKYDEPFAFQCPNGGYISGMMSRHSNKREDRIHRYFCCNVKGMTTADCKFHG
WTLYDKLFNFFVPTKRVLTGVVSIHSNKKEDRIFQFQTCKLMKRP (SEQ ID NO. 1)
Amino acid sequence of Photonin -2
MSKIELFSFLVFVAAIETSGCYCAPPKFNRRLSYCKADFTIRGLVHEYEGVEKPRGSIYLHKKYRIYP
EKFYRHLNVDFKSFHLYTPNHHSLCEKLLKVNASYIFNGKIVYDQYIINRCEWNQRWNFVPKWVKH
VLPYFYNFC (SEQ ID NO. 2)

### Red Italics Indicated Signal Peptides.

In addition, the present inventors have investigated how the signal peptides contributed to a fade-proof yellow (golden) structural coloration during conventional acid treatment.

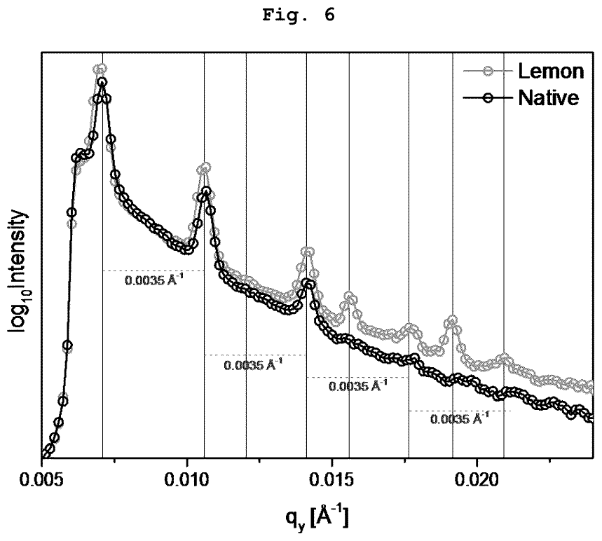

In more detail, as shown in FIGS. 6 and 7, changes in a longitudinal repeating structure were shown in Atrina byssus that was changed to gold after acid treatment (q values corresponding to the pitch were divided into 0.0035 A⁻¹, 0.0015 A⁻¹, and 0.0020 A⁻¹). Among these, when q=0.0015 A⁻¹, the pitch of the longitudinal repeating structure was 418.86 nm, and when multiplied by a general refractive index of 1.5 for an organic material, the pitch was 628.30 nm, which matched the wavelength of golden light.

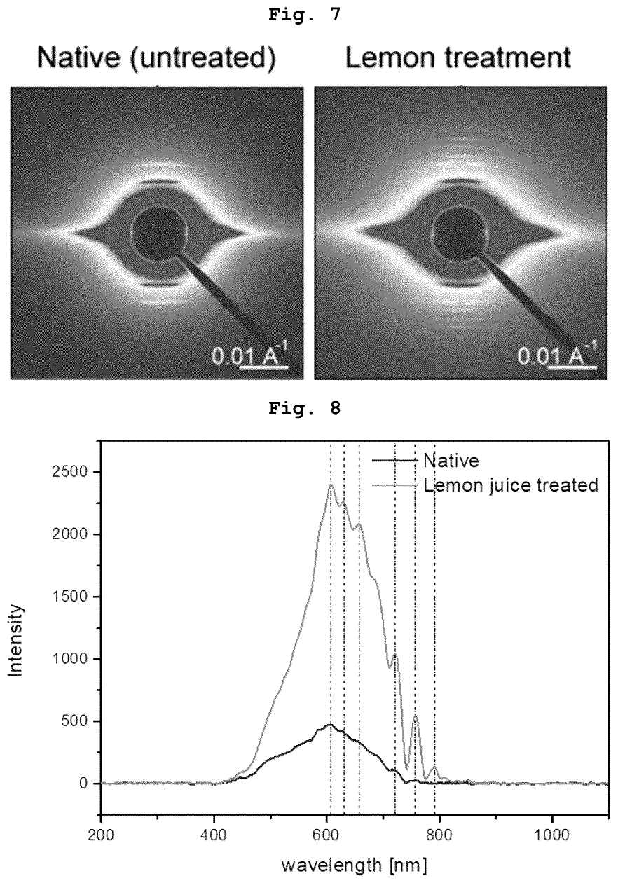

In addition, as shown in FIG. 8, after acid treatment, the Atrina byssus reflected a wavelength with a golden color, which was different from before treatment, and the corresponding wavelength (˜630 nm) matched the wavelength by the longitudinal pitch converted in FIGS. 5 and 6.

In conclusion, the Atrina sp. byssus according to the present disclosure has a spiral hierarchical structure of photonic crystal proteins that exhibit a gold color that is not changed permanently without a dye, and as a result, the Atrina sp. byssus may become silk fibers having a golden color without a dye. The golden seasilk of Atrina sp. byssus according to the present disclosure is not only safe by using ingredients that are harmless to the human body, but also creates specialty by implementing the golden color having a luxurious feeling more clearly and evenly, and thus may be preferred as a high value-added material.

### Example 3. Confirmation of Skin Improvement Effect of Seasilk Derived from Atrina sp. Byssus

In order to confirm a skin improvement effect of seasilk derived from Atrina sp. byssus, the present inventors observed cytotoxicity and cell proliferation in skin cell lines using human skin cells, HDFn cell lines.

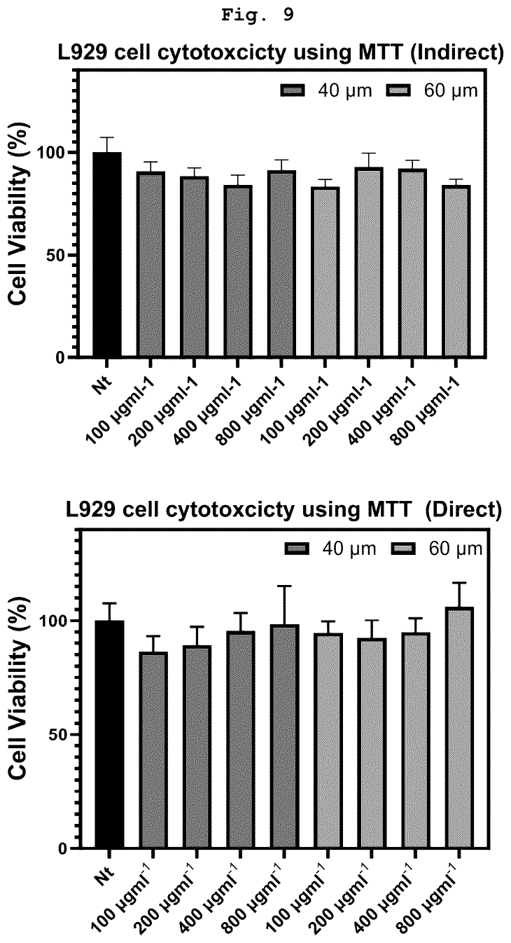

As a result, as shown in FIG. 9, the entire experimental groups (40 μm and 60 μm) showed cell viability of 80% or higher compared to a control group, which confirmed that there was no cytotoxicity.

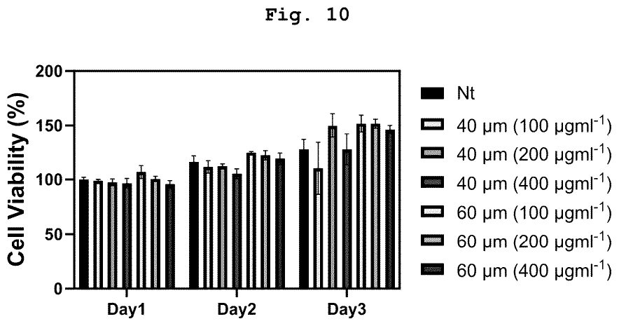

In addition, as shown in FIG. 10, as a result of observing the effect of seasilk particles having the size of 60 μm on cell proliferation of human skin fibroblasts, when 60 μm particles were treated at a concentration of 100 μg/mL on day 2, the cell viability significantly increased by 5.98% compared to a control group (p=0.021375). In addition, a significant cell proliferation effect was observed in all groups treated with 60 μm particles from day 3. More specifically, when treated with 60 μm sea silk particles at concentrations of 100 μg/mL and 200 μg/mL, respectively, the cell viability significantly increased by 23.87% (p=0.02465 for 100 μg/mL, p=0.01359 for 200 μg/mL), and when treated with a concentration of 400 μg/mL, the cell viability increased by 18.41% (p=0.03058). In the case of 40 μm particles, there was no significant increase in cell viability compared to a control group.

Hereinabove, the exemplary embodiments of the present disclosure have been described. However, various changes and modifications of the present disclosure will be made by those skilled in the art by adding, modifying, or deleting components without departing from the technical idea of the present disclosure described in the appended claims, and these changes and modifications will be within the scope of the present disclosure.

1. A method for producing a golden seasilk fiber derived from Atrina sp. byssus, comprising treating a seasilk fiber derived from Atrina sp. byssus, which is isolated from Atrina sp. byssus, with an acid solution.

2. The method of claim 1, wherein the seasilk fiber derived from Atrina sp. byssus comprises a photonic crystal Photonin-1 protein derived from Atrina sp. byssus, consisting of an amino acid sequence as set forth in SEQ ID NO: 1, and a photonic crystal Photonin-2 protein derived from Atrina sp. byssus, consisting of an amino acid sequence as set forth in SEQ ID NO: 2.

3. The method of claim 1, wherein the seasilk fiber derived from Atrina sp. byssus becomes a golden seasilk fiber having a golden structural color that does not fade due to a change in longitudinal repeating structure induced by acid treatment.

4. The method of claim 1, wherein the golden seasilk fiber derived from Atrina sp. byssus has a hierarchical structure stabilized by sugar-lectin interactions and has a structural color.

5. The method of claim 1, wherein the acidic solution is at least one selected from the group consisting of sodium citrate, acetic acid, boric acid, sorbic acid, citric acid, sodium phosphate, dibasic sodium phosphate, monobasic sodium phosphate, potassium dihydrogen phosphate, hydrochloric acid, sodium hydroxide, sodium thiosulfate, sodium sulfite, sodium sulphate, tris(hydroxymethyl)aminomethane, tris(hydroxymethyl)aminomethane hydrochloride, sodium hydrogen carbonate, sodium borate, sodium acetate, sodium bisulphate and sodium benzoate.

6. The method of claim 1, wherein the acidic solution is at least one selected from the group consisting of lemon juice, citron juice, sugar citron juice, orange juice, magnolia berry juice, bokbunja juice, wolfberry juice, cornelian cherry juice, plum juice, and vinegar.

7. The method of claim 1, wherein the seasilk fiber derived from Atrina sp. byssus and the acidic solution are treated at a ratio of 1:1000 to 1000:1 (w/w).

8. A composition for producing a golden seasilk fiber comprising the golden seasilk fiber derived from Atrina sp. byssus, produced according to the producing method of claim 1.

9. A functional cosmetic composition for improving skin, comprising a seasilk fiber derived from Atrina sp. byssus; or a golden seasilk fiber derived from Atrina sp. byssus as an active ingredient.

10. The composition of claim 9, wherein the seasilk fiber derived from Atrina sp. byssus and the golden seasilk fiber derived from Atrina sp. byssus comprise a photonic crystal Photonin-1 protein derived from Atrina sp. byssus, consisting of an amino acid sequence as set forth in SEQ ID NO: 1 and a photonic crystal Photonin-2 protein derived from Atrina sp. byssus, consisting of an amino acid sequence as set forth in SEQ ID NO: 2.

11. The composition of claim 9, wherein the seasilk fiber derived from Atrina sp. byssus becomes a golden seasilk fiber having a golden structural color that does not fade due to a change in longitudinal repeating structure induced by acid treatment.

12. The composition of claim 9, wherein the golden seasilk fiber derived from Atrina sp. byssus has a hierarchical structure stabilized by sugar-lectin interactions and has a structural color.

13. The composition of claim 9, wherein the skin improvement is selected from the group consisting of improved skin moisturizing, improved skin wrinkles, improved skin thickness, improved skin elasticity, improved skin barrier, improved skin texture, and improved pigmentation.

14. The composition of claim 9, wherein the composition has growth promoting activity of human skin fibroblasts.

15. The composition of claim 9, wherein the formulation of the cosmetic composition is at least one selected from the group consisting of a toner, a nourishing toner, a massage cream, a nourishing cream, a pack, a gel, a skin adhesive type cosmetic, a lipstick, a makeup base, a foundation, a shampoo, a rinse, a body cleanser, a soap, a lotion, an ointment, a gel, a cream, a patch, and a spray.


SEQUENCE LISTING (2 sequences)

SEQ ID NO: 1 - protein, 176 aa, Atrina sp.
MKNLQHSCLLFLVCSMISYVCEAWNNKYDGLLRFKCKTPQNHIQHILSQHDNKKEDRIYDLRCKKATGRRPSCRWTGYVNKYDEPFAFQCPNGGYISGMMSRHSNKREDRIHRYFCCNVKGMTTADCKFHGWTLYDKLFNFFVPTKRVLTGVVSIHSNKKEDRIFQFQTCKLMKRP

SEQ ID NO: 2 - protein, 144 aa, Atrina sp.
MSKIELFSFLVFVAAIAETSGCYCAPPKFNRRLSYCKADFTIRGLVHEYEGVEKPRGSIYLHKKYRIYPEKFYRHLNVDFKSFHLYTPNHHSLCEKKLKVNASYIFNGKIVYDQYIINRCEWNQRWNFVPKWVKHVLPYFYNFC